(19) United States
(12) Patent Application Publication
(10) Pub. No.: US 2025/0328063 A1
(43) Pub. Date: Oct. 23, 2025

(54) IMAGING DEVICE ADAPTOR

(71) Applicant: Keratify Limited, Sidcup Kent (GB)

(72) Inventors: Rosalind Hannen, Kent (GB); Zuber Ahmed, Kent (GB); Karl Lawrence, Kent (GB); Nihull Jakharia-Shah, Kent (GB)

(21) Appl. No.: 18/857,778

(22) PCT Filed: Apr. 19, 2023
(86) PCT No.: PCT/GB2023/051029

§ 371 Date: Oct. 17, 2024

(30) Foreign Application Priority Data
Apr. 19, 2022 (GB) 2205688.1

(51) Int. Cl. G03B 17/56; A61B 5/00; G01N 21/47; G03B 15/05

(52) U.S. Cl. CPC G03B 17/565; G01N 21/4738; G03B 15/05; A61B 5/0077; A61B 5/6898; G03B 2215/0575

(57) ABSTRACT

Aspects relate to an adaptor for use with an imaging device comprising: an elongate housing configured to be mountable over a lens of the imaging device, wherein the elongate housing comprises a first end having a first opening and a second end having a second opening; a light source positioned within the housing towards the first end; and a diffuser positioned within the housing between the light source and the second opening to provide diffuse light to the second opening. The adaptor is particularly specialised for enabling capture of high quality, repeatable, focused images of a surface of a subject.

### FIELD OF THE INVENTION

Aspects and embodiments relate to adaptors for imaging devices and, in particular, to adaptors for enabling high quality, repeatable images to be captured of a surface of a target to be imaged. Some aspects and embodiments relate to an imaging device adaptor which facilitates effective imaging of skin, either in an in vivo clinical setting or as cultured tissue in a laboratory environment. Some aspects and embodiments are configurable to support consistent sample lighting and skin imaging across an entire research and development journey from bench to clinic.

### BACKGROUND

Quantitative analysis of a surface may be performed based upon data obtained from an image of that surface. The extent to which image data from an image of a surface can be used for quantitative or qualitative analysis will depend upon the nature of an obtained image.

One surface of interest for qualitative and quantitative study comprises the surface of skin. Human skin is a highly visible organ. Fundamentals of disease diagnosis, skin-related psychological stress, use of cosmetic or aesthetic products and tracking of dermatology treatment success can be determined based upon skin appearance.

Typically, skin in a clinical setting can be imaged using a dermatoscope. A dermatoscope comprises dedicated skin imaging device. Nonetheless, it can be difficult to relate or correlate images obtained in a clinical setting to images of cultured skin tissue in a laboratory environment.

It would be desirable to provide apparatus to facilitate surface imaging, in particular skin surface imaging. Apparatus is desired which provides function in relation to surface imaging across both preclinical and clinical environments.

### SUMMARY

In a first aspect, there is provided an adaptor for use with an imaging device comprising: an elongate housing configured to be mountable over, about, around, or covering a lens of the imaging device, wherein the elongate housing comprises a first end having a first opening and a second end having a second opening; a light source positioned within the housing towards the first end; and a diffuser positioned within the housing between the light source and the second opening to provide diffuse light to the second opening.

Skin surface imaging is known in relation clinical and real world settings. Such imaging may typically be performed using a dermatoscope. However skin surface imaging is not widely adopted in relation to preclinical (ex vivo/in vitro) skin culture systems. Such skin culture systems have application in relation to monitoring of skin and tracking experimental progress. 3D skin models have typically been poor mimics of an epidermal cornified layer. Traditional ex vivo skin systems result in rapid degradation of skin surface barrier properties. As a result, images of preclinical skin samples may traditionally bear limited resemblance to what might be expected in a clinical or real world setting. Information obtained from traditional ex vivo and in vitro skin tissue applications may comprise images of that tissue obtained using a microscope or similar, and/or may depend upon a destructive imaging technique.

Emergence of physiologically relevant culture systems, for example, culture systems in which a skin tissue sample surface is exposed to atmospheric air (for example, 18-25 C, 0.03-0.05% CO₂, 35-55% relative humidity) and an underside of the skin tissue sample is cultured under appropriate temperature and gas controls (for example, 5-10% CO₂, 32-37 C, full humidity) can extend effective maintenance of ex vivo skin tissue samples, sustaining skin barrier properties for days rather than hours.

The first aspect recognises that it is possible to provide a mechanism by which an image of a skin surface can be captured both in a clinical or real world setting and in relation to appropriate preclinical models, especially those under physiologically relevant environments. Facilitating such directly comparative imaging of a skin surface can enable the development of new assays.

Imaging skin surface effectively in ex vivo and in vitro skin tissue applications can allow for comparison to clinical skin surface images. Properties which may be of interest in both ex vivo and in vitro skin tissue include: analysis which may depend upon review of skin pigmentation, skin tone and colour, inflammation, corrosion, biological efficacy, appearance of lines and wrinkles, appearance of scaring, and monitoring of in vitro wound healing.

Appropriate skin surface imaging may enable rapid product or formulation ranking. Such ranking may be performed in real time and images may be captured without damaging experimental skin tissue. Skin surface imaging can aid appropriate selection of time points during any experiment for further analysis by allowing for objective determination of when a meaningful change occurs rather than relying on a predetermined educated guess at the start of an experiment.

The first aspect recognises that there is not currently a tool to facilitate direct comparison of preclinical/laboratory skin surface analysis with similar analysis which occurs in a clinical or real world setting. Provision of such a tool may enable, for example, tracking and quantitative assessment of new products, formulations, cosmetics, drugs, injectables, and compounds throughout their developmental journey which starts in a laboratory before passing into a clinic and first human use. Such a tool could, for example, be used to help log, track and identify adverse events and immediately share this information back to R&D sites to improve toxicology and development of safer products in the future.

The first aspect provides an adaptor, sometimes referred to herein as a tool or eyepiece, which facilitates consistent surface imaging, for example skin surface imaging, using a smart phone camera. Such an adaptor allows an imaging device to be used to capture images of both ex vivo skin tissue samples and in vivo human skin under substantially identical lighting conditions. It will be appreciated that the adaptor can also be used on biological samples other than skin samples.

The first aspect may provide an adaptor for use with an imaging device. The imaging device may comprise a smartphone camera, a point and shoot camera, an SLR camera or similar digital camera or similar.

The adaptor may comprise: an elongate housing configured to be mountable over a lens of the imaging device. The elongate housing may sometimes be referred to herein as a housing, outer housing, or shroud.

The elongate housing may comprise a first end having a first opening and a second end having a second opening. The elongate housing may define a substantially cylindrical conduit between the first opening and second opening along which light may pass. The first opening may be couplable to the lens of the imaging device.

The adaptor may comprise a light source. The light source may be positioned within the housing towards the first end. The light source may be positioned adjacent to the first opening. In use, the light source may therefore be located substantially adjacent to the imaging device to which the adaptor is coupled.

The adaptor may comprise a diffuser positioned within the housing between the light source and the second opening to provide diffuse light to the second opening. The diffuser may be referred to as a pipe, light pipe, or diffuse screen. The diffuser may be configured to provide diffuse light to the second opening. That diffuse light is directed during use of the imaging device towards a sample to be imaged. Diffuse light is non-directional and therefore provides an even distribution of light to illuminate a surface evenly and with minimal shadows. In this way, the diffuser may help to provide optimal and repeatable conditions for imaging a surface.

The first aspect recognises that provision of consistent and diffuse light to a surface to be imaged allows for capture of an image of the surface in which image artefacts attributable to shadowing, glare or similar may be substantially avoided.

The camera eyepiece tool in accordance with the first aspect can enable provision of consistent lighting to a skin surface image from preclinical (in vitro, ex vivo, laboratory) images through to clinical/in human images. As a result, image data can be quantified, stored and compared across the entire R&D lifecycle of any skin care, for example, therapeutic, cosmetic or aesthetic, product.

Adaptors in accordance with the first aspect may provide for parameters such as lighting, focal distance, image processing and quantification to be consistent between skin surface images.

The elongate housing may direct and/or focus light from the light source towards the second opening and therefore surface to be imaged. The elongate housing may help create diffuse light. The elongate housing may prevent light from the light source escaping the adaptor. The elongate housing may block external light not produced by the light source from reaching a sample surface to be imaged.

The light source may be positioned between the centre of the elongate housing and the first end. By positioning the light source to be remote from the second end, light emitted by the light source has a greater length of adaptor along which to be diffused before exiting the adaptor at the second opening. Positioning the light source between the centre of the elongate housing and the second end may result in the light source being positioned so close to the second opening that light emitted from the light source would not be sufficiently diffuse on reaching the second opening. Inappropriate positioning of the light source within the adaptor may lead to creation of intensity ‘hotspots’ where the light is concentrated. Such inadequately scattered light may cause undesirable artefacts on the sample and/or in a captured image.

An adaptor in accordance with the first aspect can provide a cheap, easy to manufacture, simple and compact tool for adapting an existing imaging device to produce repeatable high quality surface images. The adaptor provides an imaging tool which can support skin surface imaging in both “real” and “artificial” skin contexts.

The adaptor may be dimensioned to maintain a minimum distance between the imaging device and a sample to be imaged, wherein the minimum distance is approximately equal to a minimum focal length of the imaging device. Accordingly, the adaptor facilitates a 1:1 magnification of images. 1:1 imaging allows an image to be formed on an image sensor of the imaging device sensor that is the same size as the sample being imaged. Using a 1:1 magnification produces sufficiently high quality images for analysing skin samples. The resulting images are likely to have high resolution and good scalability. Provision of an arrangement in which the adaptor acts to maintain or force capture of an image when the imaging device is a reproducible fixed distance from the sample can be beneficial in relation to reproducibility and consistency of captured images. Such spacing control between operational components of the imaging device and a target surface to be imaged can obviate a need for additional lenses or magnification. It will be appreciated that the length of the adaptor may be selected in dependence upon intended imaging device and/or intended sample type.

In embodiments, a 1:1 magnification may not be used and the minimum distance may facilitate an alternative magnification.

The adaptor may be dimensioned to maintain the minimum distance within a specific tolerance, for example, 5 mm, or more preferably 1 mm.

As described above, it is envisaged that the first aspect may facilitate collection of images of skin surface samples. In particular, the first aspect may facilitate image capture in relation to real human skin (on the body); a portion of skin taken from a human; or a cultured or laboratory produced skin tissue sample. In use, an adaptor in accordance with the first aspect is configured to set or maintain a distance between a sample and the imaging device. A portion of the adaptor may directly abut or contact the sample itself, for example, when imaging skin in vivo. Alternatively, a portion of the adaptor may engage with a holder which contains a skin sample to be imaged. In this case, the depth/distance added to the distance between the sample and the imaging device by the holder can be taken into account by the adaptor. Such variation of use may be accounted for using an interface cuff as discussed in more detail below.

The elongate housing may comprise an inner surface. At least a portion of the inner surface may be configured to reflect visible light. The inner surface may comprise a reflective material or finish such that visible light is reflected within the housing. A reflective inner surface may prevent light emitted by the light source escaping the adaptor and may help direct light towards the second opening.

The at least a portion of the inner surface configured to reflect visible light may be configured to provide a diffuse reflection of light. Accordingly, light emitted from the light source within the adaptor is diffused via two mechanisms: diffuse reflection from at least a portion of an inner surface of the housing and diffuse transmission and reflection provided by the diffuser. Provision of two diffusion mechanisms can improve diffusion of light within the adaptor, thereby improving consistency of output illumination for imaging. Provision of an adaptor which provides two light diffusion mechanisms can enable provision of a compact adaptor, since at least one dimension may be fixed, without undue sacrifice of light diffusion performance.

At least a portion of the inner surface between the first opening and the second opening may comprise a tapered portion. The taper may comprise a bottleneck shape or a curved substantially frustoconical shape. Provision of a narrowing portion or taper which narrows towards the second end, can allow light emitted by the light source to be generally directionally guided towards the second opening. Such a taper or equivalent shaping may direct or focus light emitted by the light source towards the surface of the sample to be imaged. The taper may be located to help create diffuse light at the second opening by reflecting light towards the diffuser and the centre of the adaptor. This is particularly the case in embodiments in which an inner surface is provided having a smooth bottleneck shape without any sharp or flat edges.

At least a portion of the inner surface is white. Provision of a white inner surface can aid more complete or effective reflection of a full spectrum of visible light wavelengths from the inner surface and assist in providing appropriate light to a surface to be imaged.

At least a portion of the inner surface may comprise a textured finish. The textured finish may comprise a ridged, sandblasted, or beadblasted finish. In other words, the inner surface may comprise a broken, rough, serrated, or uneven surface portion. Textured finishes can scatter light upon reflection or transmission. That is to say, textured reflective surfaces result in light which is reflected in unpredictable directions, which can help to create diffuse light. Textured finishes also help reduce glare.

The elongate housing may be opaque. Accordingly, light from the light source is prevented from escaping the adaptor through the housing. Similarly, light from external sources is not let into the adaptor through the housing.

The elongate housing may be formed from a plastics material. The plastics material may comprise polycarbonate. Forming the housing from plastics material may result in a light and manoeuvrable adaptor.

At least a portion of the inner surface may be configured to reflect infrared or ultraviolet light. Some embodiments may comprise special adaptations to enhance IR or UV imaging. For example, the inner surface may comprise an aluminium surface to help reflect UV light. Such an aluminium surface may be coated, for example, by paint or other similar layer, or may have a metallic finish.

The diffuser may extend between the first opening and the second opening. Provision of a diffuser which extends the entire length of the adaptor or housing may provide for improved diffusion because the light has more opportunity to interact with the diffuser.

The diffuser may comprise a pipe or substantially cylindrical element or member located coaxially within the elongate housing. Such an arrangement may compliment implementations in which the light source comprises a ring light arrangement discussed in more detail below.

At least a portion of the diffuser may be translucent such that light passing through the translucent portion is scattered to produce a diffuse light. At least a portion of the diffuser that is translucent may be frosted. At least a portion of an outside surface of the diffuser facing the inner surface of the elongate housing is partially reflective, allowing for reflection of light between the outer surface of the diffuser and the inner surface of the elongate housing. Such reflection between components may allow for creation of more diffuse light at the second opening.

The diffuser may comprise an inner surface. At least a portion of the inner surface may comprise a textured finish. The textured finish may comprise a ridged, sandblasted, or beadblasted finish.

The light source may comprise a ring light. A ring light can help provide multidirectional and evenly distributed light which can be diffused by the components of the adaptor to provide even illumination of a sample to be imaged and mitigate shadows on the surface to be imaged.

The ring light may comprise a plurality of circumferentially spaced light sources. The plurality of circumferentially spaced light sources comprises at least one LED. In embodiments, the plurality of circumferentially spaced light sources comprises at least four LEDs. The plurality of circumferentially spaced light sources may comprise at least six LEDs. The plurality of light sources may be individually controllable. Accordingly, if more directional lighting is required, or a different light intensity is required, the light source may be configurable to support provision of a range of lighting conditions to a sample.

The ring light may comprise a single light source. The single light source may comprise a tube light or light-pipe. The light source may comprise a fibre optic light source. The light source may comprise a Chip on Board (COB) “spotless” or “seamless” lighting device.

Whilst it is generally preferable to provide an even illumination, by using a single light source radially offset from the centre of the adaptor (which may be an individually controlled light source of a plurality of light sources), custom lighting or shading may be supported.

The ring light may be positioned coaxially within the housing to help provide an even illumination around the adaptor.

The light source may comprise of one or more switch to control the light source.

The light source may be positioned radially outwardly of the diffuse screen.

The light source may be configured to produce light in the visible part of the spectrum. The light source may be configured to replicate the intensity and wavelength distribution of natural light. The light source may be configured to produce infrared or ultraviolet light.

The adaptor may comprise: an interface cuff configured to be removably couplable to the second end of the elongate housing over the second opening. The interface cuff may be dimensioned to maintain focus of the imaging device on a sample provided at the end of the adaptor. An inner surface of an interface cuff may be reflective and shaped to direct light incident upon the inner surface towards the second opening of the adaptor.

The interface cuff may be configured to be directly engageable with, or directly abut, a sample surface to be imaged. Accordingly, the imaging device and the sample to be imaged are maintained at the minimum distance. By engaging the adaptor, or adaptor including interface cuff, with the sample, a repeatable distance equivalent to the minimum distance can be created between a sample surface to be imaged and the imaging device. Consistent positioning when taking multiple images over a period of time or across a range of samples results in capture of consistent image data. Furthermore, such capture can allow the images taken using the adaptor to be directly compared to each other despite possible variations in an imaging scenario.

The interface cuff may be configured to be directly engageable with a holder configured to hold a sample to be imaged such that the imaging device and the sample to be imaged are maintained at the minimum distance. By engaging the adaptor with the holder, a repeatable distance equivalent to the minimum distance can be created between the sample and the imaging device. This allows consistent positioning when taking multiple images over a period of time or across a range of samples. This allows the images taken using the adaptor to be comparable to each other despite the varying situation.

It will be appreciated that interface cuffs of different dimensions may be required to acquire images at the desired minimum distance for samples for which the adaptor engages directly and samples which the adaptor engages a holder. In this way, the adaptor can facilitate direct comparisons between images of in vivo and in vitro samples. The interface cuff may be extendable to accommodate for the different scenarios. For example, the interface cuff may comprise a telescopic extending portion.

Examples of possible sample holders with which the adaptor may engage by means of an appropriate interface cuff include: a culture unit, a tissue culture plate, a Franz diffusion cell, a transwell, an organ/skin-on-chip device and a fluidic device.

The interface cuff may comprise an image hole, wherein the image hole is dimensioned to set the area of the sample to be imaged. For example, a large image hole may allow for a larger image to be captured and vice versa.

The interface cuff may comprise a curved portion configured to present a curved surface to the imaging device. Sharp or flat edges within the adaptor can impede focusing of the imaging device, i.e., the imaging device will focus on a feature of the adaptor and not on the sample. By presenting a curved surface to the imaging device, flat edges are avoided and interruption of the focus of the imaging device can be mitigated.

A portion of an outer surface of an image cuff may be dimensioned or shaped to form a friction fit engagement with an opening provided in the sample holder. The interface cuff may be opaque. Accordingly, external light may be prevented from entering the adaptor and light emitted by the light source escaping the adaptor. Furthermore, the lighting environment provided to a sample located in the holder may be fully controlled by the adaptor.

The interface cuff can be interchanged to help ensure the correct focal distance for imaging skin in a clinic/on humans or in a preclinical skin culture system. The interface cuffs allow the adaptor to be used in relation to a range of cell culture methods, for example, tissue culture well plates, transwells, flow cells, organ-on-chip models.

The interface cuff may comprise a portion having a complimentary shape to an engagement portion of a sample holder.

The interface cuff may be positioned such that when it is coupled to the second end of the elongate housing, at least a portion of the interface cuff is positioned radially between the diffuser and the elongate housing. The adaptor may comprise a spacing collar positioned between the light source and the diffuser. The collar can help retain the light source and/or diffuser in position within the elongate housing.

The adaptor may consist of: the elongate housing, the light source, the diffuser, the collar and the interface cuff. An adaptor according to the first aspect may provide high quality, consistent images of a sample surface and may be used to image skin in both in vivo and in vitro applications without the need for any additional lenses or mirrors. This design can provide a cheap and easy to manufacture adaptor which does not compromise on resulting quality of captured image.

In some embodiments, the adaptor further comprises: a focus ring supported over the second opening, wherein the focus ring comprises at least one focussing feature extending radially within the focus ring to define a focus plane, wherein the at least one focussing feature is configured to allow the imaging device to focus on the focus plane, and wherein, in use, the at least one focussing feature is positioned adjacent to a sample to be imaged.

Such an arrangement recognises that imaging devices used for imaging skin samples sometimes struggle to focus on the skin samples. Images of skin samples which are not in focus may not be suited to use for analysis and can lead to inefficient analysis since repeat images may be required. Whilst it may be envisaged that use of an imaging device with a more reliable autofocus could be beneficial, some arrangements as described herein can provide a simple mechanical solution to an automatic focusing problem.

According to some arrangements, the at least one focussing feature may be configured to focus the imaging device on a plane defined by the at least one focussing feature. Given that, in use, the at least one focussing feature is arrangeable to be adjacent to the skin sample, the imaging device can be forced to focus correctly in a plane suitable for imaging a skin sample surface. Accordingly, in focus images can be reliably captured using such an arrangement. Furthermore, provision of a focus ring may support use of a range of different imaging devices to successfully capture in focus images regardless of the quality and reliability of their autofocus.

In some embodiments, the at least one focussing feature comprises: a protrusion arranged to extend radially into the second opening. In some embodiments, the focussing feature comprises a tab, flap, or similar. In some embodiments, the focussing feature is formed from a resilient material, having a rigidity selected such that it generally holds a position extending radially into the second opening, but pliable enough to deform to substantially conform to align with a surface of the sample to be imaged.

In some embodiments, the at least one focussing feature comprises: a plurality of bristles. Bristles may be flexible so they flex upon contact with, for example, a skin sample. This can avoid excess pressure being exerted on a sample, thereby reducing the likelihood of damaging the sample. Furthermore, in contrast to a fixed focal length arrangement, in which no accommodation is made for irregularities in a sample to be imaged, it can allow the focus ring to accommodate, for example, for varying thicknesses of skin sample. Provision of flexible bristles can allow bristles to flex and thereby to stay immediately adjacent to a surface of a sample to be imaged. In the context of skin imaging, an ability to focus on a sample surface can be particularly useful because in-focus images can be captured even as a skin sample thickness changes over time.

In some embodiments, the at least one focussing feature is made from a biologically compatible material. In some embodiments, the at least one focussing feature is made from a chemically inert material. In some embodiments, the at least one focussing feature may be formed from a biologically compatible material. In some embodiments, the at least one focussing feature is made from a biologically compatible plastic such as PTFE, PP or HDPE. In some embodiments, the at least one focussing feature is formed from a biocompatible or non-biocompatible material with a biocompatible coating. In some embodiments, the coating and/or the material being coated are chemically inert. This can simplify the manufacturing process if the focussing feature also provides a reference colour for colour correction because the colour may be applied during the coating process.

In some embodiments, the at least one focussing feature is a different colour to the housing. In some embodiments, the at least one focussing feature is a different colour to the plug discussed below. In some embodiments, the at least one focussing feature comprises at least one reference colour visible, in use, to the imaging device. The reference colour or colours may, for example, be used as a colour calibration feature. Such colour calibration may comprise a coloured focussing feature having a colour or colours selected from, for example, a red, green, blue (RGB) selection or a cyan, yellow, magenta (CYM) palette, or any other appropriate known colour. In this way, such embodiments may support colour correction or calibration across one or more captured images, since the focussing feature can provide a known reference colour forming part of a captured image which can be used for calibration purposes.

In some embodiments, the at least one focussing feature comprises: a plurality of focussing features. Having more than one focussing feature may help focus the imaging device on the focus plane. It may also allow smaller focussing features to be used compared to an arrangement on which only a single focussing feature is provided. In some embodiments, the at least one focussing feature or formation comprises at least 3, at least 5, or at least 6 focussing features or formations.

In some embodiments, the plurality of focussing features are evenly distributed around the focus ring. In this way, the focussing features may aid the imaging device to reliably focus on the focus plane. In some embodiments, the focussing features are distributed in a non-uniform manner. By providing a non-uniform distribution of focussing features, more distributions are available, thereby enhancing the ability of the focussing features to provide a unique signature for a particular plug. Similarly, non-uniform distributions can enhance use of the focussing features as an alignment tool for orientating images in post-production because a non-uniform distribution of focussing features may only align in one orientation whereas an even distribution of focussing features may align in many orientations.

In some embodiments, a first feature of the plurality of focussing features is a first colour and a second feature of the plurality of focussing features is a second colour, the second colour being different from the first colour. In this way, the focussing features can provide a unique signature which can be recognised in images taken to identify which focus ring was used. The user may wish to use the same equipment for imaging a particular skin sample over the course of an experiment. The signature provided by such colour coding helps the user to ensure the same equipment is being used for each imaging session. This could improve the consistency of images which is beneficial when comparing images.

In some embodiments, the adaptor further comprises: a plug configured to couple the housing to a sample holder, wherein the focus ring is supported by the plug. The plug can help guide and position the adaptor within a well of a sample holder. Providing the focus ring within the plug allows it to be positioned next to a sample held within the well of the sample holder, thereby helping the camera focus on the sample. It will be appreciated that the focus ring could alternatively be supported by an interface cuff, for example, one as defined in the first aspect, where no plug is present.

In some embodiments, the focus ring is slidably supported by the plug. In some embodiments, the plug defines a track configured to retain the focus ring yet permit the focus ring to slide along the plug. In this way, the focus ring can be allowed to slide along the plug to accommodate a sample being imaged. As discussed above, the bristles may flex to accommodate a sample and to maintain the focussing features adjacent the surface to be imaged of the sample. Additionally or alternatively, the focus ring may be slidably mounted within the plug such that, in use, it may move to maintain its position adjacent the surface to be imaged of the sample and avoid damaging the sample. This can facilitate imaging a sample which grows and thickens over the course of an experiment because the focus ring can slide to ensure the focussing features define the focus plane adjacent the surface of the sample regardless of the thickness of the sample.

In some embodiments, the plug is removably couplable to the second end of the housing. This may allow the plug to be inserted into the well of the sample holder before being coupled to the housing of the adaptor.

In some embodiments, the second end of the housing comprises: an integral interface cuff, wherein the plug is configured to be couplable to the integral interface cuff. This may simplify the manufacture of the adaptor.

In some embodiments, the plug comprises: a locking formation configured to mate with a corresponding locking formation defined by the housing such that the housing is rotationally locked relative to the plug when the locking formations are mated. In this way, the orientation between the housing and the plug is consistent between uses. Provision of a rotational lock may further prevent the orientation from changing whilst images are being captured.

In some embodiments, the plug comprises a second locking formation configured to mate with a corresponding locking formation defined by the sample holder such that the adaptor is rotationally locked relative to the holder when the locking formations are mated. In this way, the orientation between the adaptor and the sample holder is consistent between uses. The rotational lock prevents the orientation from changing whilst images are being captured. In combination with the locking features between the plug and the housing, the adaptor allows images to be captured consistently from the same orientation relative to the sample. This may facilitate image analysis and comparison of images.

According to a further aspect, there is provided: an adaptor for use with an imaging device, the adaptor comprising: a focus ring, wherein the focus ring comprises at least one focussing feature extending radially within the focus ring to define a focus plane, wherein the at least one focussing feature is configured to allow the imaging device to focus on the focus plane, and wherein, in use, the at least one focussing feature is positioned adjacent to a sample to be imaged.

In some embodiments, the adaptor comprises: an elongate housing configured to be mountable over, about, around, or covering a lens of the imaging device, wherein the elongate housing comprises a first end having a first opening and a second end having a second opening; a light source positioned within the housing towards the first end; and a diffuser positioned within the housing between the light source and the second opening to provide diffuse light to the second opening.

According to a second aspect, there is provided a kit comprising the adaptor of the first aspect and a mount configured to mount the adaptor to the imaging device.

The mount may be configured to removably mount the adaptor to the imaging device. The mount may comprise a clamping member. The mount may comprise a pipe clamp. The mount may comprise an adhesive portion for attaching the mount to the imaging device. The mount may comprise a magnetic portion configured to couple to an imaging device, and/or cooperating magnetic portions located on the adaptor and mount to couple the adaptor and mount. The mount may comprise a clip configured to couple to an imaging device. The mount and/or adaptor may comprise cooperating clip portions located on the adaptor and mount to couple the adaptor and mount. The mount may be configured to couple to the imaging device and/or the adaptor by means of a friction fit.

When the adaptor is mounted to the imaging device, light may not enter the elongate housing through the first opening. This facilitates creating the controlled environment for imaging a sample and ensures sample lighting is controlled by the components of the adaptor.

The kit may comprise: the imaging device. In embodiments, the imaging device is a mobile phone.

The kit may further comprise the holder configured to hold at least one sample to be imaged. The holder may comprise at least one of: a culture unit, a tissue culture plate, a Franz diffusion cell, a transwell, an organ/skin-on-chip device and a fluidic device.

In embodiments, the kit comprises a skin clamp or plug configured to retain skin in a position to be imaged whilst being captured in vivo.

According to a third aspect, there is provided a method of providing an adaptor for an imaging device, comprising: configuring an elongate housing to be mountable over a lens of the imaging device, wherein the elongate housing comprises a first end having a first opening and a second end having a second opening; positioning a light source within the housing towards the first end; positioning a diffuser within the housing between the light source and the second opening to provide diffuse light to the second opening.

According to a fourth aspect, there is provided a method of using an adaptor according to the first aspect with an imaging device to capture an image of a sample, comprising: mounting the adaptor to the imaging device; engaging a distal end of the adaptor with the sample or a holder containing the sample; and capturing an image using the imaging device.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 shows an exploded view of an adaptor according to an arrangement;

FIG. 2 shows a section through an adaptor according to an arrangement;

FIG. 3 shows an imaging device, a mount and an adaptor according to an arrangement;

FIG. 4a shows an adaptor according to an arrangement within a mount;

FIGS. 4b and 4c show sections through an adaptor according to an arrangement;

FIGS. 5a, b and c show sections through an adaptor according to an arrangement each comprising an interface cuff having an image hole with a different diameter;

FIG. 6 shows an adaptor according to an arrangement together with a holder; and

FIG. 7 comprises a selection of in vivo skin surface images captured using an imaging device and adaptor according to an arrangement and example image analysis performed on the captured skin images;

FIG. 8 shows an imaging device, a mount and an adaptor according to a second embodiment;

FIG. 9 shows an exploded view of the apparatus of FIG. 8;

FIG. 10 shows a cross-section through the apparatus of FIG. 8;

FIG. 11 shows an end view of the adaptor of FIG. 8;

FIG. 12 shows a perspective view of the apparatus of FIG. 8;

FIG. 13 shows images of skin samples taken using an adaptor according to the embodiment of FIG. 8;

FIG. 14 shows an adaptor according to a third embodiment; and

FIG. 15 shows the embodiment of FIG. 14 engaging a sample holder.

### DESCRIPTION OF THE EMBODIMENTS

Before discussing the embodiments in any more detail, first an overview will be provided.

Arrangements described provide an adaptor for use with an imaging device. The adaptor may comprise an elongate housing configured to be mountable over a lens of the imaging device. The elongate housing may comprise a first end having a first opening and a second end having a second opening. The adaptor may comprise a light source positioned within the housing towards the first end of the elongate housing. The adaptor may comprise a diffuser positioned within the elongate housing between the light source and the second opening to provide diffuse light to the second opening.

The adaptor according to described arrangements is dimensioned to maintain a minimum distance between the imaging device and a sample to be imaged. The minimum distance is equal to a minimum focal length of the imaging device such that a 1:1 magnification is obtained. Such selection of the dimensions of the adaptor can aid capture of a sample image that is the same size as the sample being imaged on a sensor of the imaging device. Such an image is scalable and high resolution.

In described arrangements, portions of the housing can be reflective and/or opaque to provide an isolated and controlled lighting environment inside the adaptor which is directly applied to a sample to be imaged. The adaptor of described arrangements may have a housing including a reflective inner surface with a bottleneck shape which operates to direct light towards the second opening. The second opening is, during use of the adaptor with the imaging device, directed towards the sample to be imaged.

In described arrangements, the light source comprises a ring light to provide an even illumination to a sample surface to be imaged.

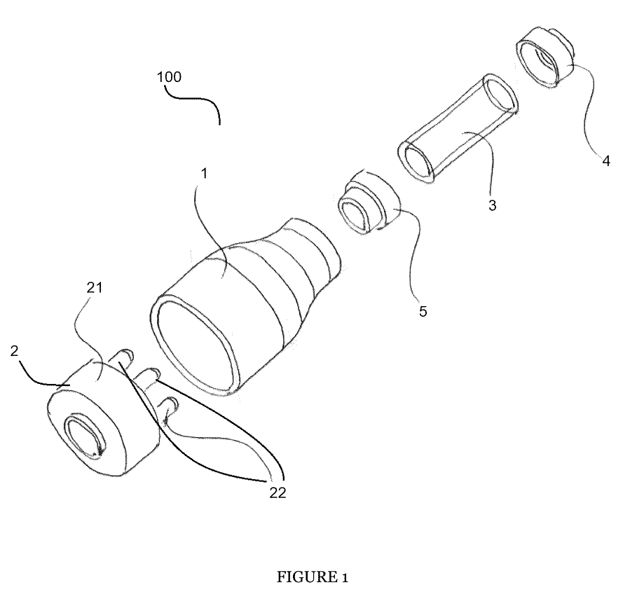

FIG. 1 shows the constituent parts of an adaptor 100 according to an arrangement. The adaptor 100 comprises: an elongate housing 1, a light source 2, a diffuser 3, an interface cuff 4, and a collar 5.

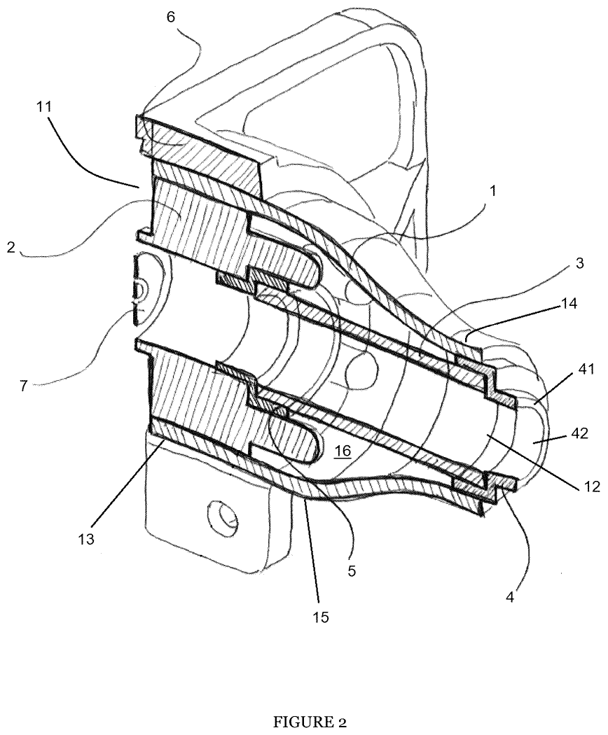

FIG. 2 shows the arrangement of the components of the adaptor 100 when assembled. The housing 1 is elongate and comprises a first end 13 having a first opening 11 and a second end 14 having a second opening 12. The housing 1 in the arrangement shown reduces in diameter between the first end and the second end. The housing 1 is, in the arrangement shown, formed from a material which is opaque to visible light.

The housing 1 comprises an inner surface 16. The inner surface includes a narrowing portion or taper 15. The taper narrows in the direction of the second end 14. The taper may be curved such that the housing has an inner surface which comprises a substantially bottleneck shaped portion. The inner surface 16 has a reflective and textured finish. The textured finish may be a ridged, sandblasted, or beadblasted finish which helps produce diffuse light.

The light source 2 in the arrangement shown comprises a ring light. The ring light comprises a base 21 and a plurality of LEDs 22. It will be appreciated that any suitable alternative ring light may be used, for example, alternative arrangements may be formed using a ring light which includes fibre optic light sources or appropriately shaped tube lighting. The light source may comprise a Chip on Board (COB) “spotless” or “seamless” lighting device.

The light source 2 is positioned within the housing 1 towards the first end 13. The light source 2 is coaxial with the housing 1. The base 21 may sit within the first opening 11 as shown in the embodiment of FIG. 2. In alternative arrangements, the housing 1 may substantially enclose the light source on two or three sides.

The diffuser 3 of the arrangement shown comprises a generally cylindrical tube or light pipe positioned coaxially within the housing 1. The diffuser 3 is positioned between the light source 2 and the second opening 12 such that light from the light source 2 must pass through the diffuser 3 before travelling through the second opening 12.

The diffuser 3 is configured to diffuse light travelling through the diffuser 3 by scattering the light. The scattered light may be light transmitted through the diffuser 3 or reflected by the diffuser 3. In the arrangement shown the diffuser 3 comprises a translucent light pipe. The diffuser 3 can be made from a plastics material. The diffuser 3 comprises an inside and an outside surface. One or both of these surfaces may have a textured finish and/or be partially reflective.

The interface cuff 4 comprises an annular shape and is configured to couple to the housing 1 over the second opening 12. The interface cuff comprises a surface 41 for directly engaging either a sample surface to be imaged, for example, the skin on a human, or a holder containing a sample surface to be imaged.

Together with the housing 1, the interface cuff 4 defines the total length of the adaptor 100. It will be appreciated that provision of a longer interface cuff may provide a longer adaptor. The provision of one or more interface cuff 4 may therefore allow for customisation of the overall length of the adaptor 100.

The interface cuff 4 of the arrangement illustrated further comprises an image hole 42. The diameter of the image hole helps determine the size of the sample being imaged. The interface cuff 4 may comprise a staggered portion or narrowing portion to define the image hole 42. The staggered portion may comprise a curved portion (not shown in the figures) which presents a curved surface towards the first opening 11 such that no flat edges are presented towards the first opening 11 of the housing 1.

The interface cuff 4 can be made from a plastics material and may couple to the housing 100 via a push fit mechanism or any other suitable connection mechanism. A portion of the interface cuff 4 may be positioned radially between the housing 1 and the diffuser 3 when coupled to the housing 1. The interface cuff 4 may have a textured finish interior surface to reduce glare.

The collar 5 of the illustrated arrangements is configured to help position and retain the light source 2 and the diffuser 3 within the housing 1. The collar 5 is coaxial with the housing 1. A portion of the collar 5 is positioned between the light source 2 and the diffuser 3. It will be appreciated that an adaptor 100 according to alternative arrangements may not require a collar 5 and that the diffuser 3 and light source 2 may be attached directly to each other.

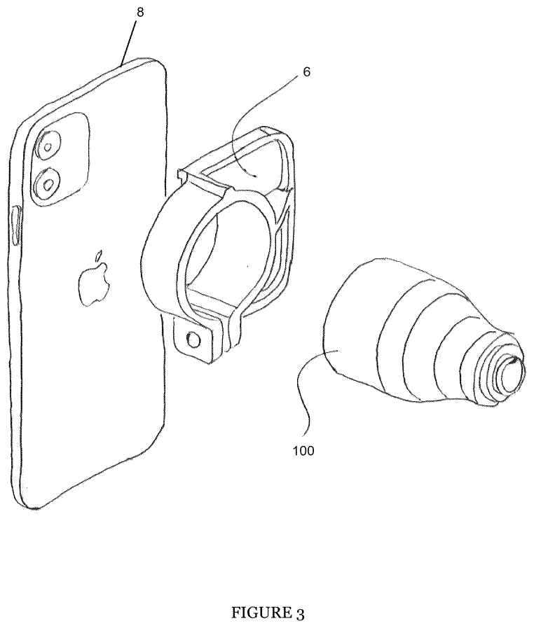

The adaptor 100 of illustrated arrangements is configured to be used with a mount 6 seen best in FIG. 3. The mount 6 is configured to removably couple the adaptor 100 to an imaging device 8, for example, a mobile phone with built in camera. The mount 6 may comprise a pipe grip to hold the outer surface of the substantially cylindrical housing of the adaptor 100 and an adhesive portion to couple the mount 6 to the imaging device 8. The mount may be made from a plastics material. It will be appreciated that any suitable mount may couple the adaptor 100 to the imaging device 8.

In use, the adaptor 100 is mounted to the imaging device 8 over a lens 7 of the imaging device 8 (shown in FIG. 2) using the mount 6. The mount 6 is coupled to the first end 13 of the adaptor. The mount 6 and the adaptor 100 are configured such that no light can enter into the adaptor 100 through the first opening 11. Any flash of the imaging device is turned off or may be covered by the adaptor 100 such that light cannot enter through the first opening 11. In this way, the lighting inside the adaptor 100 which is provided to a surface to be imaged by the imaging device is controlled by the components of the adaptor.

The second end 14 of the adaptor in accordance with illustrated arrangements is, in use, directed towards a sample surface to be imaged. In the arrangements shown, the engagement surface 41 of the interface cuff 4 is abutted against the skin or a holder containing the sample to be imaged. The interface cuff 4 may be coupled to the housing 1 before or after mounting the adaptor 100 to the imaging device 8. The interface cuff may be replaced with another interface cuff whilst the adaptor is still mounted to the imaging device. Accordingly, the same adaptor can be used with various imaging cuffs selected in dependence upon the nature of the sample surface to be imaged.

The light source 2 emits light into the housing 1. It will be appreciated that the light source may be switched on at any time prior to an image being captured by an imaging device to which the adaptor is coupled or may be activated at the same time an image is captured.

The light is directed by the reflective inner surface 16 of the housing towards the second opening 12 and the image hole 42. The reflective inner surface 16 facilitates guiding the light from the light source towards a sample surface to be imaged with minimal losses. The opaque nature of the housing 1 and the interface cuff 4 prevents light from escaping the adaptor 100 and unwanted light entering the adaptor 100 through the housing 1. The bottleneck shape of the inner reflective surface of the housing 1 encourages reflections towards the second opening 12. The textured finish to the inner surface 16 helps create diffuse light and reduces glare.

Any light from the light source 2 which reaches the second opening 12 has also passed through diffuser 3. The diffuser is positioned between the light source 2 and the second opening 12. A translucent diffuser, for example, a frosted diffuser, may be used to scatter/diffuse light transmitted through the diffuser. A textured finish on the inside and/or outside surface of the diffuser may further facilitate diffusion. Provision of a diffuse reflective surface as the inner surface of the housing and a diffuse light pipe ensures that the adaptor provides lighting to a surface of the sample to be imaged which is diffuse and not directional. Such light avoids creation of unwanted shadow on the surface to be imaged. The use of diffuse light to illuminate a sample surface to be imaged allows for creation of a repeatable, high quality, and situation transferrable environment in which an appropriate surface image can be captured.

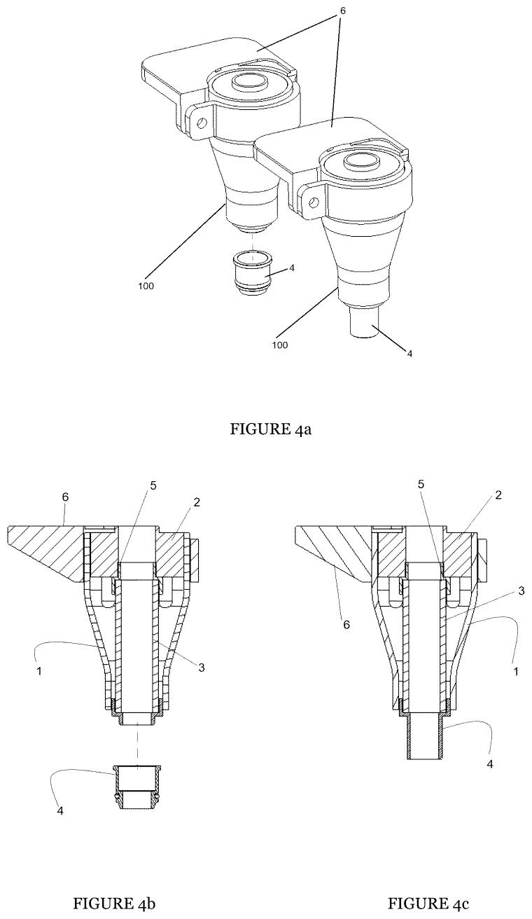

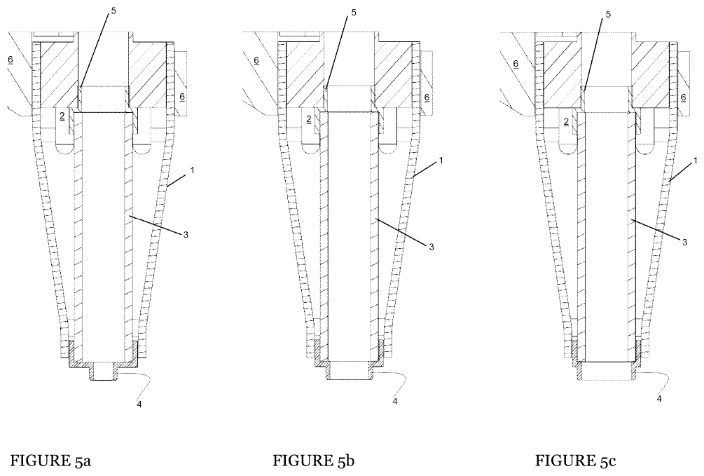

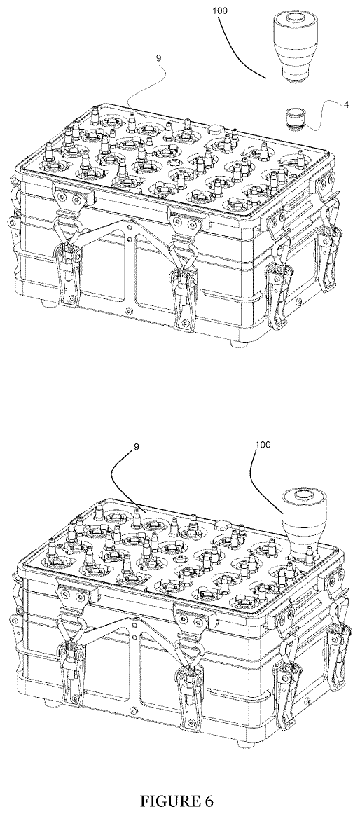

The adaptor 100 may be provided in a kit. The kit may comprise the adaptor 100 and the mount 6. The kit may comprise an imaging device 8. The kit may comprise at least one holder 9 as shown in FIG. 6. For example, the holder may comprise a culture unit, a tissue culture plate, a Franz diffusion cell, a transwell, an organ/skin-on-chip device or a fluidic device. The kit may comprise multiple different interface cuffs. For example, the kit may include interface cuffs with different lengths as shown in FIGS. 4b and 4c. Alternatively or additionally, the kit may comprise interface cuffs with differently sized image holes as shown in FIGS. 5a, b and c. One of the interface cuffs may be configured for use directly on a human skin surface and another interface cuff may be configured for use with a holder of an ex vivo skin or tissue sample.

Arrangements provide a method of adapting an imaging device, comprising: configuring an elongate housing to be mountable over a lens of the imaging device, wherein the elongate housing comprises a first end having a first opening and a second end having a second opening; positioning a light source within the housing towards the first end; positioning a diffuser within the housing between the light source and the second opening to provide diffuse light to the second opening.

Arrangements provide a method of using an adaptor 100 with an imaging device 8 to capture an image of a sample, comprising: mounting the adaptor to the imaging device; engaging a distal end of the adaptor with the sample or a holder containing the sample; and capturing an image using the imaging device.

In some arrangements, features of the adaptor or eyepiece include: a mount to connect to a smart phone or camera. The arrangement illustrated comprises: a mount designed for use with an iPhone 12 mini but the mount can be adapted to fit other phones or cameras, an LED ring light, an internal frosted light pipe/tube to ensure even diffuse lighting across the skin surface, an external shroud (housing) that links to the eyepiece mount to prevent external (non-controlled) light from entering the system. Arrangements described comprise an interface cuff designed to fit into the well of a culture system (as shown), or with an interface cuff designed to rest on the surface of human skin, for example, in a clinic or personal setting. The difference between an interface cuff for the well of a culture system vs human use is primarily length of the cuff, where a longer cuff is required to ensure the correct focal distance directly on human skin, and a shorter cuff is required to accommodate the additional length of the well skin holding. The eyepiece can be fully sterilized, for example by autoclave, so that it can be used under aseptic conditions required for tissue culture.

The current eyepiece houses an LED ring light source, but other light sources across the electromagnetic spectrum (such as UV), could be used to assess other properties of the skin/skin surface. It may also be possible to include single light sources, for example, at a 45 degree angle to assess skin surface textural changes. The length of the eyepiece has been developed for sharp focus at 1:1 magnification, but could be shortened to an appropriate length/focusing distance for greater magnification, for example is using a macro lens.

The integration with a smart phone in the described arrangement, particularly an iPhone, was chosen over an (DSLR) camera because such an imaging device enables rapid focus, has a small camera lens that has proved to be suitable for this form of eyepiece to focus on a single culture well, the potential for automated time-lapse imaging and automatic photo loading via a wifi/internet connection to a cloud-based image software processing suite (SeeVivo). Images in RAW file formats can be collected as well as images automatically processed by the smart phone. Skin image files captured using the adaptor of described arrangements can be processed and quantified. Such quantification may comprise, for example, quantification of colour channels (RGB or greyscale). Appropriate algorithms may be applied to captured image data to quantify, for example, one or more skin surface parameter, including those related to edge, line and/or textural detection. All image data collected via use of an adaptor in accordance with described arrangements can be quantified and compared against baseline values from the same untreated skin donor/sample or broader database.

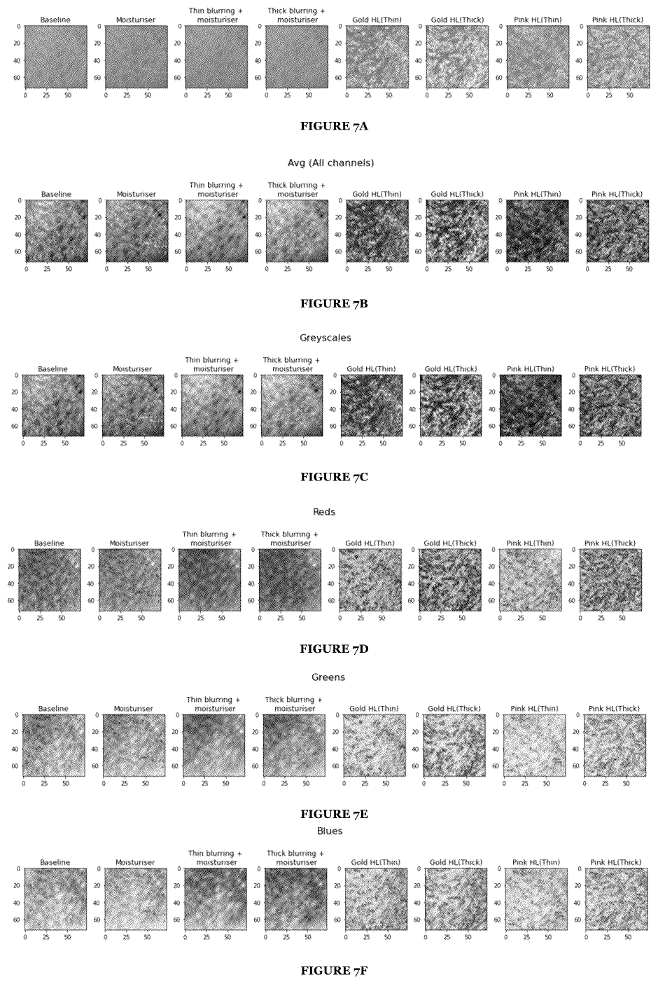

FIG. 7 comprises a selection of in vivo skin surface images captured using an imaging device and adaptor according to an arrangement and example image analysis performed on the captured skin images.

FIG. 7 shows a selection of skin surface images of the type which can be collected using the adaptor described herein. The captured image data can be used to assess changes to skin surface properties after, for example, application of cosmetic products. All images shown in FIG. 7 are from a single female facelift skin donor with visible pores. Baseline (untreated skin) was then applied with a moisturiser, a skin blurring product, a gold highlighter or pink highlighter. FIG. 7A shows unprocessed skin images captured using an adaptor according to described arrangements. The image data of the images of FIG. 7A can be processed to provide an image comprising a total channel image (all pixels) as shown in FIG. 7B; a greyscale image (pixels distributed according to the human eye) as shown in FIG. 7C, a red channel image (red pixels only) as shown in FIG. 7D, a green channel image (green pixels only) as shown in FIG. 7E, and/or a blue channel image (blue pixels only) as shown in FIG. 7F.

The image data obtainable via use of an adaptor as described may support quantified analysis of a skin surface. For example, analysis of pixel intensity distribution for each channel can be presented graphically in histograms and quantified relative to the baseline (untreated) skin image. Peak shifts in pixel intensity either to the right (corresponding to brighter) or left (corresponding to darker) of baseline peak intensity, can be used to determine which product creates the “brightest” or “darkest” skin image in each channel. The data handling is conducted in a manner to enable product ranking, with single value outputs.

Captured image data may also provide a quantitative overall distribution of colour which can be mapped, for example, using violin plots to provide clear graphical representation of skin surface analysis to support product ranking.

Further analysis of each channel can be conducted using algorithms relating to bright lines/edges, as well as texture to assess pixel uniformity.

The arrangements of the first aspect allow for the quantitative assessment of skin surface characteristics to be obtained in relation to the skin surface directly and cultured skin surface samples, and, more particularly, the provision of a controlled lighting and image capture environment allow for direct comparison between images captured of ex vivo/in vitro skin surface samples and in vivo skin sample images.

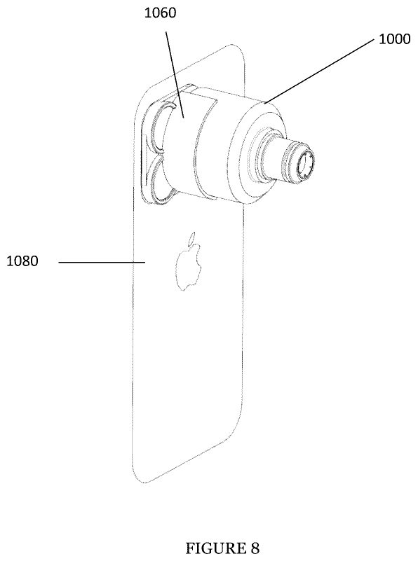

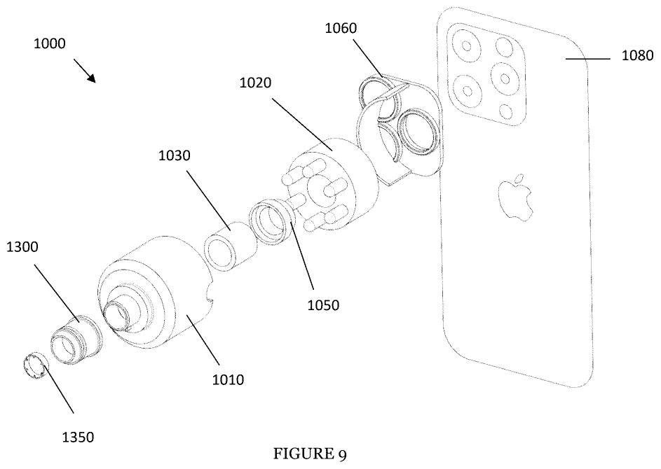

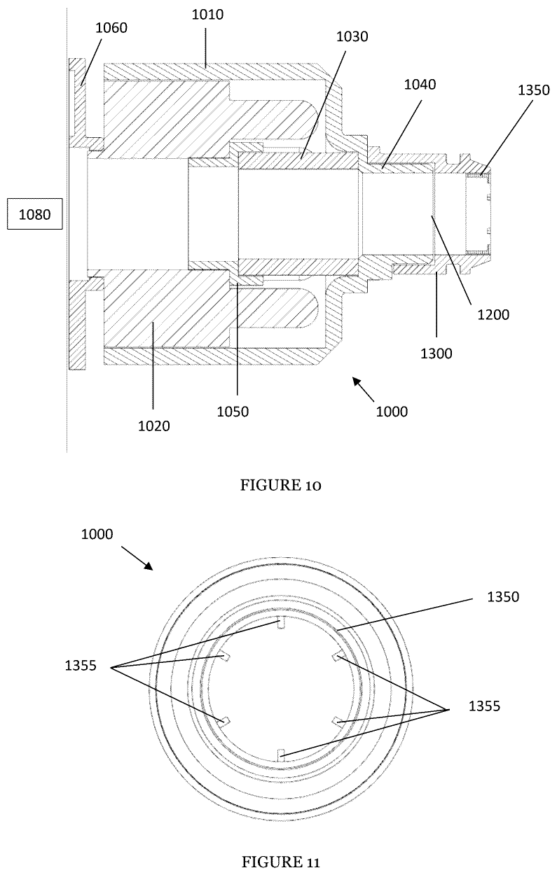

FIG. 8 shows another example embodiment of an adaptor 1000. The adaptor 1000 is similar to the adaptor 100 described above in that the adaptor 1000 is configured to be mounted on an imaging device 1080 using a mount 1060. The adaptor 1000 may couple to the mount 1060, for example, via a friction fit. Similarly, the mount 1060 may couple to the imaging device 1080, for example, via a friction fit. FIG. 9 shows an exploded view of the apparatus of FIG. 8. The adaptor 1000 comprises: a housing 1010, a light source 1020, a diffuser 1030, an interface cuff 1040 (shown in FIG. 10), a collar 1050, a plug 1300, and a focus ring 1350.

FIG. 10 shows that the components of adaptor 1000 fit together in substantially the same way as for the adaptor 100 described above. This embodiment differs in that the interface cuff 1040 is integrally formed with the housing 1010. This reduces the number of loose components and simplifies the manufacture of the adaptor because fewer components need to be made. Moreover, the tolerances required when manufacturing a push fit interface cuff, such as for interface cuff 4, are small and so manufacture needs to be precise. By integrating the interface cuff 1040 with the housing 1010, there are no corresponding tolerances to consider during manufacture.

Another difference is that the inner surface of the housing 1010 is not tapered but instead comprises a stepped shape. A ledge defined in the stepped inner surface of the housing 1010 helps support the diffuser 1030. Whilst a tapered shape helps guide light to the second opening to help ensure sufficient light for imaging reaches the second opening, the adaptor 1000 obviates the need for a tapered housing because it is shorter in length than the adaptor 100. In this way, sufficient light will reach the second opening of the adaptor even without a tapered or curved inner surface. Accordingly, the adaptor 100 is more compact and has reduced materials cost. In some embodiments, the adaptor is less than or equal to 50 mm in length including the length provided by the plug 1300.

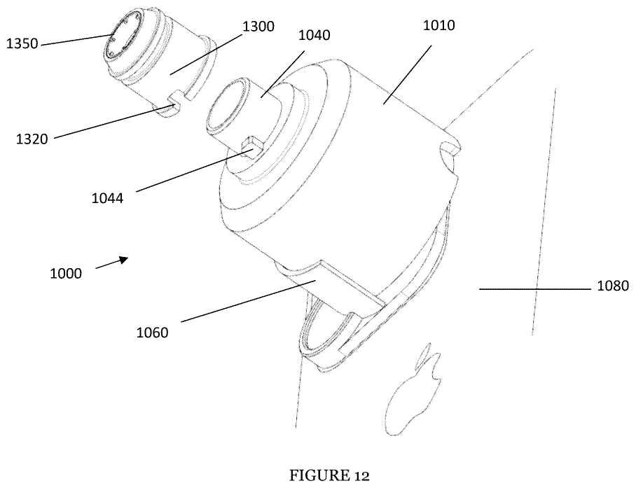

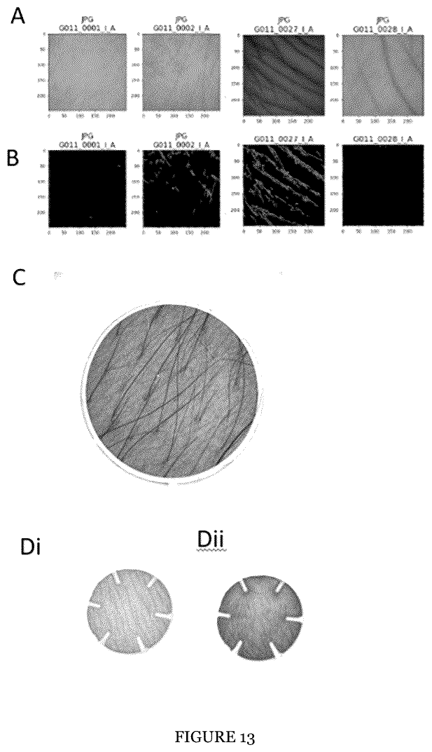

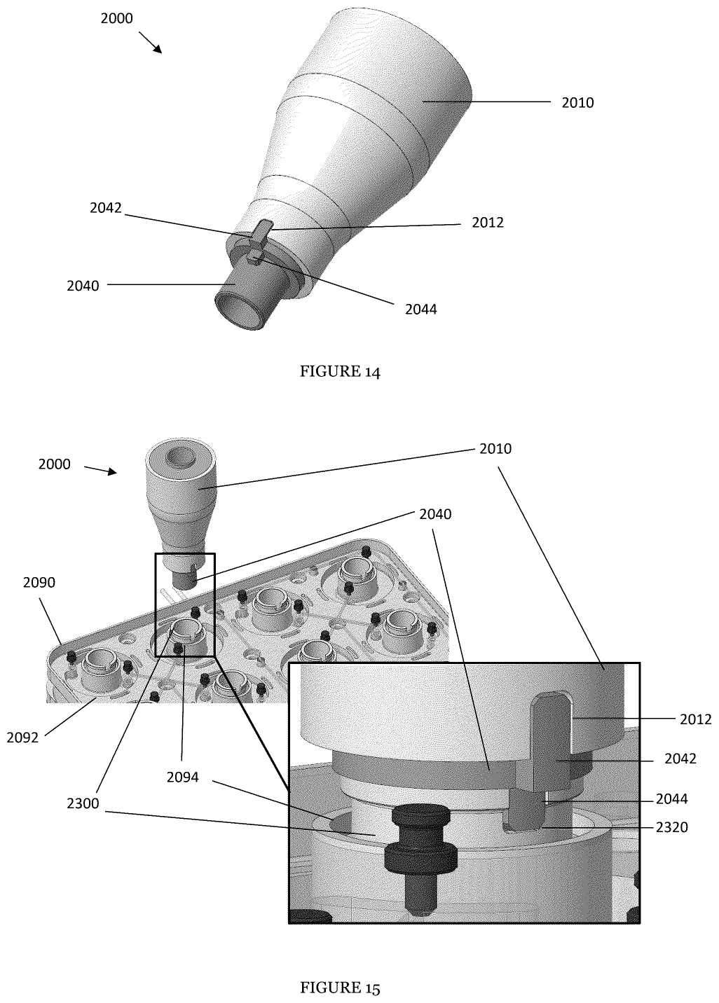

The plug 1300 is configured to couple the housing 1010 to a sample holder (e.g., holder 2090 shown in FIG. 14). The plug 1300 is configured to removably couple to the housing 1010 by receiving the integrated interface cuff 1040. The plug 1300 is designed to allow the user to place the adaptor 1000 in a repeatable position within a sample holder. FIG. 12 shows that the housing 1010 and the plug 1300 comprise corresponding mating formations 1320, 1044 configured to interlock with each other. The formations 1320, 1044 are configured to permit the housing 1010 to mate with the plug 1300 in a longitudinal direction but only at a particular orientation. When mated, the housing 1010 of the adaptor 1000 is rotationally locked relative to plug 1300. The plug 1300 may comprise a second locking formation for interlocking with a corresponding formation of the sample holder (this may be similar to locking formation 2044 shown in FIGS. 13 and 14 described in more detail below). These ‘poka-yoke’ features allow the orientation of the adaptor 1000 to be identical each time the adaptor 1000 is used. Accordingly, images taken using the adaptor 1000 are consistent which can facilitate image analysis and the comparison of images.

As best shown in FIGS. 10 and 11, the plug 1300 supports a focus ring 1350 over the second opening 1200 defined by the housing 1010 such that light from the light source 1020 passes through the focus ring 1350. The focus ring 1350 comprises at least one focussing feature extending within the focus ring 1350 to define a focus plane. The at least one focussing feature is configured to help the imaging device focus in the focus plane. In use, the focussing features are positioned adjacent to a surface to be imaged of a biological sample, such as a skin sample, such that the focus plane is immediately adjacent to the surface being imaged. In this way, the imaging device can be forced to focus in a plane suitable for imaging the surface of the sample. In this embodiment, the at least one focussing feature comprises six groups of bristles 1355 evenly distributed circumferentially about the focus ring 1350. The bristles 1355 are flexible such that, when placed adjacent to the sample, they can accommodate for variations in the thickness of the sample by flexing. Such flexing can also help avoid exerting excess pressure on the sample which could cause damage. The bristles 1355 are therefore able to adapt to the thickness of a sample (which is likely to change during the course of an experiment) to consistently define the focus plane immediately adjacent to the surface of the sample. Thus, the bristles 1355 help the imaging device 1000 focus in a plane suitable for imaging the surface of the sample. This is particularly advantageous when imaging skin samples ex vivo because the size and thickness of skin samples may change over time.

It will be appreciated that any size, distribution and number of focussing features may be used provided that the arrangement is capable of attracting the focus of the imaging device 1080. In one embodiment, the focus ring has a diameter of 10 mm and the at least one focussing feature is less than or equal to 400 microns wide, less than or equal to 400 microns deep and less than or equal to 1.25 mm long. In some embodiments, the at least one focussing feature is between 200 and 500 microns wide, preferably approximately 400 microns wide. In some embodiments, the at least one focussing feature is between 0.5 and 2 mm long, preferably approximately 1.25 mm microns long. In some embodiments, the diameter of the focus ring is at least one of 3 mm, 5 mm or 10 mm. In some embodiments, the length of the at least one focussing feature is less than or equal to 12.5% of the diameter of the focus ring, preferably less than 7.5%. In this way, the one or more focussing feature does not impose too much upon the surface being imaged. The dimensions of the focussing features may be scaled with the diameter of the focus ring to help ensure the focussing features do not obstruct a sample being imaged. It will be appreciated that the focussing features could be any size providing they do not substantially obscure the surface being imaged. Furthermore, the focussing features need not all be the same size. In some embodiments, the focussing features vary in size.

Whilst FIG. 11 shows that the focussing features are evenly distributed circumferentially about the focus ring, this need not be the case. Instead, the circumferential distribution of the focussing features may be non-uniform. The focussing features can be configured to provide a signature that allows the focus ring to be identified within images taken using that focus ring. In this way, the focussing features may provide a code for identifying a plug. To this end, providing a plurality of focussing feature with a non-uniform distribution can provide greater variation in signatures compared to an even distribution. It is envisioned that an adaptor may be provided with multiple plugs each comprising a different distribution of focussing features. This can help a user identify which focus ring was used to take a particular image meaning a user can ensure the same focus ring is used, for example, throughout the course of an experiment for consistency. Additionally or alternatively, the signature provided by the distribution of focussing features can be used for identifying individual patients to ensure there is no mix-up of data.

The focussing features may further provide an alignment function. During post-production, focussing features can be aligned in images taken throughout an experiment to obtain images of consistent orientation which can facilitate the comparison and analysis of images. Providing a plurality of focussing features with a non-uniform distribution can facilitate this function because an even distribution may align at multiple orientations (is rotationally symmetric) whereas a non-uniform distribution may only align in one orientation (is not rotationally symmetric).

The focus ring 1350 need not be supported in a fixed manner as shown in FIG. 10. In some embodiments, the focus ring is slidably mounted to the plug such that the entire focus ring can slide along the plug away from and towards the surface being imaged. The plug may comprise a track within which the focus ring is supported. The track may permit the focus ring to slide along the plug but also retain the focus ring within the plug. In some embodiments, the plug comprises a biasing means configured to bias the focus ring in the direction of the sample to be imaged. In use, the focus ring is positioned over the sample to be imaged. When imaging in the well of a sample holder, the plug may have a predefined position within the well at which images should be captured. For example, the plug may be in position when rotational locking formations of the plug and the holder have mated. Given that the position of the plug may remain constant but the thickness of the sample may vary over time, the relative position between the adaptor and the sample can change. Accordingly, when positioning the adaptor and the plug, the sample may press against the focus ring to move it along the track. In this way, the focussing features can be maintained immediately adjacent to the surface of the sample to be imaged regardless of the relative positioning between the adaptor and the surface being imaged. When the adaptor is removed from the imaging position, the focus ring is moved back to its original position by the biasing means.

Furthermore, one or more of the focussing features may be coloured differently from the plug 1300 and/or the housing 1010. In some embodiments, at least one of the focussing features comprises a reference colour, visible to the imaging device in use. Use of colour in this way may aid colour correction of images captured using the adaptor 1000. Additionally, the colour of the formations 1355 may be used to provide a signature for that focus ring 1350 and the plug 1300 supporting the focus ring 1350. In this way, the focus ring 1350 and plug 1300 used to capture an image may be identified from the image. Where there are multiple focussing features, a particular combination of colours may provide the signature.

It will be appreciated that the focus ring 1350 need not be supported by the plug, but instead may be supported by the interface cuff 1040 over the second opening 1200. In this case, the at least one focussing feature may be aligned with the second opening 1200. It will be appreciated that the interface cuff may be integrated or removable in such an embodiment.

The housing 1010, the plug 1300, the focus ring 1350 and the bristles 1355 are made from a biocompatible material and preferably a chemically inert material. In some embodiments, the focus ring 1350 and the bristles 1355 are made from a biologically compatible plastic such as PTFE, PP or HDPE. In some embodiments, the focus ring 1350 and the bristles 1355 are formed from a non-biocompatible material with a biocompatible coating. This can simplify the manufacturing process if the focussing feature also provides a reference colour for colour correction because the colour may be applied during the coating process. In some embodiments, the focussing features are coated specifically to provide a reference colour. Coatings of the focussing features according to some embodiments are chemically compatible.

In use, the adaptor 1000 is mounted to the imaging device 1080 using the mount 1060. The imaging device 1080 may be, for example, an iPhone 14 Pro Max, although it is envisioned that other imaging devices may be used. Preferably, the imaging device has an automatically adjustable focal length. The housing 1010 is then coupled to the plug 1300 such that the at least one focussing feature is positioned over the second opening 1200. The plug 1300 may be mounted directly on the housing 1010 before positioning the adaptor 1000 on a sample holder or, alternatively, the plug 1300 may already be inserted within a well of a sample holder with the housing 1010 subsequently being inserted into the plug 1300. If the plug 1300 is coupled to the housing 1010 before being inserted into the sample holder, the adaptor 1000 is then positioned on the holder. The coupling between the housing 1010 and the plug 1300 and between the adaptor 1000 and the sample holder is complete when the locking formations have mated. At this stage, the imaging device 1080 is in position to capture images of samples held within the well of the sample holder. After capturing the desired images, the adaptor 1000 can be removed from the holder and the plug 1300 removed. Alternatively, the plug 1300 may be left in the well of the sample holder. When imaging directly on human skin in vivo, the plug is coupled to the housing and then the adaptor is positioned over the surface to be imaged such that the focussing feature of the focus ring are positioned immediately adjacent to the surface to be imaged. In some embodiments where there is no plug, the focus ring may be coupled directly on the housing such that in use the focussing features are positioned immediately adjacent to the sample being imaged.

FIG. 13 shows use of a bristle focus ring for achieving a focal plane on human skin. FIG. 13A shows images captured using an adaptor without a bristle ring. The images show that focusing on the skin was more difficult to achieve with lighter coloured skin and skin without strong features such as wrinkles. FIG. 13B shows the images generated using canny edge detection from the images in FIG. 13A. It can be seen that line and edge detection was unable to detect features of lighter coloured skin when the original images were not in focus. FIG. 13C shows an image taken of lighter coloured skin with hairs. The focus ring was developed after the observation that the focus on lighter coloured skin was clearer when hairs were present, suggesting the hair helped create the right automated focal plane. FIG. 13D shows an example image captured using a bristle focus ring. The focus ring created a good focal plane on the surface of the skin leading to an in focus image which can be used in subsequent analysis. Note that images of skin from the same donor generated by the automated post-image processing of the Apple iPhone 14 ProMax can include a blue tinge (shown in FIG. 13Di) relative to the RAW image (shown in FIG. 13Dii). The use of one or more coloured bristles in the bristle focus ring could therefore be used as a reference to check colour consistency in images, or could be used to rebalance colour levels from RAW images. In FIG. 13Dii, some depressions on the skin from a prototype bristle focus ring can be seen highlighting that the bristles need to be flexible and/or the bristle ring height adjustable to prevent excess pressure on the skin surface that could interfere with the imaging data.

FIG. 14 shows another embodiment of an adaptor 2010. The adaptor 2000 comprises a housing 2010 and a removable interface cuff 2040. The interface cuff comprises two locking formations: a locking formation 2042 configured to mate with a corresponding locking formation 2012 defined by the housing 2010, and a locking formation 2044 configured to mate with a corresponding locking formation 2320 defined in a well 2094 of the holder 2090 as shown in FIG. 15. These locking formations allow the adaptor to be easily inserted and removed from the sample holder yet provide a rotational lock between the housing 2010, the interface cuff 2040 and the holder 2090 when mated. In this way, the adaptor 2000 ensures that an imaging device has a consistent and repeatable orientation relative to a sample held by the sample holder 2090. This may facilitate image analysis and image comparison.

FIG. 15 shows a close-up view of the adaptor engaging a well 2094 of the sample holder 2090. The well is defined in a top surface 2092 of the holder. It envisioned that the plug 1300 of the embodiment shown in FIGS. 8 to 12 may comprise a similar locking feature to formation 2044 for locking with a sample holder.

The apparatus of all embodiments described above may be provided in a kit comprising any combination and any number of the components described. For example, a kit according to an embodiment may comprise an adaptor with more than one plug. A kit according to an embodiment may comprise an adaptor and a mount. A kit according to an embodiment may comprise an adaptor and a holder. A kit according to an embodiment may comprise an adaptor, one or more plugs and a holder. A kit according to an embodiment may comprise an adaptor, a mount, and one or more plugs.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

### REFERENCE SIGNS

1, 1010, 2010 Housing
2, 1020 Light Source
3, 1030 Diffuser
4, 1040, 2040 Interface Cuff
5, 1050 Collar
6, 1060 Mount
7 Lens
8, 1080 Imaging Device
9, 2090 Holder
11 First Opening
12, 1200 Second Opening
13 First End
14 Second End
15 Taper
16 Inner Surface
21 Base
22 LEDs
41 Engagement Surface
42 Image Hole
100, 1000, 2000 Adaptor
1300 Plug
1350 Focus Ring
1355 Focussing Features
1320, 1044 Interlocking Formations
2012, 2042 Interlocking Formations
2044, 2320 Interlocking Formations
2092 Top Surface of Holder
2094 Well

1. An adaptor for use with an imaging device comprising:
an elongate housing configured to be mountable over a lens of the imaging device, wherein the elongate housing comprises a first end having a first opening and a second end having a second opening;
a light source positioned within the elongate housing towards the first end; and
a diffuser positioned within the elongate housing between the light source and the second opening to provide diffuse light to the second opening.

2. The adaptor of claim 1, wherein the adaptor is dimensioned to maintain a minimum distance between the imaging device and a sample to be imaged, wherein the minimum distance is approximately equal to a minimum focal length of the imaging device.

3. The adaptor of claim 1, wherein the elongate housing further comprises an inner surface configured to reflect visible light.

4. (canceled)

5. The adaptor of claim 3, wherein at least a portion of the inner surface configured to reflect visible light provides a diffuse reflection.

6. The adaptor of claim 3, wherein at least a portion of the inner surface between the first opening and the second opening comprises a taper having a bottleneck shape or a curved frustoconical shape.

7. (canceled)

8. The adaptor of claim 3, wherein at least a portion of the inner surface is white and comprises a textured finish, wherein the textured finish comprises a ridged, sandblasted, or beadblasted finish.

9. (canceled)

10. The adaptor of claim 1, wherein the elongate housing is opaque.

11. The adaptor of claim 1, wherein;
the diffuser extends between the first opening and the second opening; and
the diffuser comprises a pipe located coaxially within the elongate housing.

12. (canceled)

13. The adaptor of claim 1, wherein the diffuser comprises a translucent portion such that light passing through the translucent portion is scattered to produce a diffuse light.

14. The adaptor of claim 1, wherein the light source comprises a ring light, the ring light comprising a plurality of circumferentially spaced light sources that are individually controllable.

15. (canceled)

16. (canceled)

17. (canceled)

18. The adaptor of claim 1, wherein the adaptor further comprises an interface cuff configured to be removably couplable to the second end of the elongate housing over the second opening.

19. The adaptor according to claim 18, wherein the interface cuff is configured to be directly engageable to a sample to be imaged such that the imaging device and the sample to be imaged are maintained at a minimum distance.

20. The adaptor according to claim 18, wherein the interface cuff is configured to be directly engageable with a holder configured to hold a sample to be imaged such that the imaging device and the sample to be imaged are maintained at a minimum distance.

21. (canceled)

22. The adaptor according to claim 18, wherein the interface cuff comprises a curved portion configured to present a curved surface to the imaging device.

23. (canceled)

24. The adaptor according to claim 1, further comprising a focus ring supported over the second opening, wherein the focus ring comprises at least one focusing feature extending radially within the focus ring to define a focus plane, wherein the at least one focusing feature is configured to allow the imaging device to focus on the focus plane, and wherein in use the at least one focusing feature is positioned adjacent to a sample to be imaged.

25. The adaptor according to claim 24, wherein the at least one focusing feature comprises at least one of: a plurality of bristles, a color different from the elongate housing, or a plurality of focusing features.

26. (canceled)

27. (canceled)

28. (canceled)

29. The adaptor according to claim 25, wherein a first feature of the plurality of focusing features is a first color and a second feature of the plurality of focusing features is a second color, the second color being different from the first color.

30. The adaptor according to claim 24, further comprising a plug configured to couple the elongate housing to a sample holder, wherein the focus ring is slidably supported by the plug.

31. (canceled)

32. (canceled)

33. The adaptor according to claim 30, wherein the plug comprises:
a first locking formation configured to mate with a first corresponding locking formation defined by the elongate housing such that the elongate housing is rotationally locked relative to the plug when the first locking formation is mated with the first corresponding locking formation; and
a second locking formation configured to mate with a second corresponding locking formation defined by the sample holder such that the adaptor is rotationally locked relative to the sample holder when the second locking formation is mated with the second corresponding locking formation.

34. (canceled)

35. (canceled)

36. (canceled)

37. A method of providing an adaptor for an imaging device, comprising:
configuring an elongate housing to be mountable over a lens of the imaging device, wherein the elongate housing comprises a first end having a first opening and a second end having a second opening;
positioning a light source within the elongate housing towards the first end;
positioning a diffuser within the elongate housing between the light source and the second opening to provide diffuse light to the second opening.
